(19) United States
(12) Patent Application Publication
(10) Pub. No.: US 2022/0192941 A1
(43) Pub. Date: Jun. 23, 2022

(54) SKIN REJUVENATION

(71) Applicant: L'OREAL, Paris (FR)

(72) Inventors: Rebecca CHEN, Princeton, NJ (US); Anne-Laure Suzanne BERNARD, New York, NY (US); Xi YAN, Berkeley Heights, NJ (US); Janet WANGARI-TALBOT, Clark, NJ (US)

(73) Assignee: L'OREAL, Paris (FR)

(21) Appl. No.: 17/132,830

(22) Filed: Dec. 23, 2020

(51) Int. Cl. A61K 8/22; A61K 8/42; A61K 8/81; A61K 8/31; A61K 8/9794; A61K 8/34; A61K 8/73; A61K 8/86; A61K 8/368; A61K 8/60; A61K 8/55; A61K 8/19; A61K 8/24; A61K 8/41; A61Q 19/10; A61Q 19/08; A61Q 19/00; A61Q 19/02; A61K 8/92

(52) U.S. Cl. CPC A61K 8/22; A61K 2800/28; A61K 8/8176; A61K 8/31; A61K 8/9794; A61K 8/342; A61K 8/731; A61K 8/8147; A61K 8/8158; A61K 8/73; A61K 8/86; A61K 8/368; A61K 8/602; A61K 8/55; A61K 8/19; A61K 8/24; A61K 8/41; A61Q 19/10; A61Q 19/08; A61Q 19/008; A61Q 19/02; A61K 8/92; A61K 2800/522; A61K 2800/48; A61K 8/42

(57) ABSTRACT

A cosmetic composition for exfoliating skin and method for exfoliating skin using the cosmetic composition are provided. The cosmetic composition has at least one oxidizing agent; at least one fatty compound present from about at least 20 wt. %, the said fatty compound having a molecular weight of less than about 400 g/mol; and wherein the composition can accelerate skin rejuvenation, reduce skin pore size appearance and casual sebum, improve skin tone evenness and radiance, and improve overall skin appearance.

### FIELD OF THE INVENTION

The instant disclosure relates to cosmetic compositions for exfoliating skin and methods for treating skin, for example, methods for exfoliation of the skin of the face.

### BACKGROUND OF THE INVENTION

Skin renewal can be stimulated by removal of the outer keratinous layer. Such removal can be effected mechanically, for example, by rubbing with an abrasive composition, or chemically. Chemical peeling uses active chemical agents that interact with the complex structure of the skin, removing the outer layer (stratum corneum for superficial peels, epidermis and possibly dermis for medium and deep peels) to induce a controlled injury to the skin and stimulate skin regeneration. The regenerated skin is smoother and appears younger. Peeling is commonly used to treat visible and/or tactile irregularities of the human skin, to attenuate defects of pigmentation such as skin discoloration and the marks due to acne or varicella, and to smooth irregularities in the texture, in particular fine lines and wrinkles.

Various acids have been used as the active chemical agents for chemical peels. Common acids include α-hydroxy acids (AHA, such as glycolic acid), β-hydroxy acid (BHA, salicylic acid for example), tricholoracetic acid (TCA) and retinoic acid. TCA and retinoic acid are usually used in medium to deep peels and performed in dermatologist office or in a medical spa setting. AHA and BHA can be used as home peels or superficial peels and medium or deep peels depending on the concentration. These acids deliver skin benefits via interacting with the skin to accelerate desquamation and stimulate new skin cell growth.

Higher concentrations of acids improve the efficacy of a peel but often cause discomfort and irritation (e.g., reddening, tingling, burning sensation), and require longer healing times. Even superficial peels cause burning sensations and excessive skin peeling that can last for days. Medium and deep peels are more intense. Recovery from a medium or deep peel generally requires staying indoors for multiple days, and patients must wait several months before receiving a subsequent treatment. Also, normal AHA/BHA exfoliation composition can cause irritation and hyperpigmentation problem for skin of color.

It is an object of the instant disclosure to provide a skin care composition that overcomes at least one of the aforementioned drawbacks associated with products that employ high concentration of acids and provides good skin-brightening efficacy. Yet another object of the instant disclosure is to demonstrate a progressive increase in skin-brightening efficacy when applied regularly.

### SUMMARY OF THE DISCLOSURE

The compositions and methods hereof are characterized, in various embodiments, as comprising an oxidizing agent, in some embodiments comprising hydrogen peroxide and fatty compound, and includes fatty compounds at a range from at least about 20% by weight, to impart on the skin rejuvenation of the skin. The inventors discovered that the combination of some oxidizing agent with some low molecular weight fatty compounds enables cosmetic compositions to accelerate the rejuvenation process of the skin.

Cosmetic compositions according to an aspect of the disclosure typically include:
a. At least one oxidizing agent;
b. at least one fatty compound present from about at least 20 wt. %, the said fatty compound having a molecular weight of less than about 400 g/mol; and
wherein the composition can accelerate skin rejuvenation, reduce skin pore size appearance and casual sebum, improve skin tone evenness and radiance, and improve overall skin appearance; and
wherein all percentages by weight are based on the total weight of the cosmetic composition.

In some instances, the cosmetic composition is formulated without the presence of an activator for the at least one oxidizing agent.

The methods of the instant disclosure include applying the cosmetic composition at least once a week for at least about 1 minutes, then rinse off the skin. When applied repeatedly over time to keratinous tissue, an acceleration of the skin rejuvenation can be observed.

In an exemplary embodiment, a cosmetic composition may include at least one oxidizing agent present in an amount of from about 1 to about 4 wt. % based on the total weight of the cosmetic composition. In some embodiments, the at least one oxidizing agent is chosen from a peroxide or a metal peroxide. In some particular embodiments, the least one oxidizing agent is chosen from hydrogen peroxide, urea peroxide, carbamide peroxide, and PVP hydrogen peroxide (a complex of polyvinylpyrrolidone and hydrogen peroxide) and metal peroxide combinations thereof. In some embodiments, the oxidizing agent is hydrogen peroxide. In some embodiments, the peroxide is a metal peroxide selected from sodium peroxide, calcium peroxide, barium peroxide, magnesium, lithium and zinc.

In accordance with some of the various embodiments, the at least fatty compound is selected from low molecular linear or branched alkane (C6-21 alkane): isohexadecane, C8-9 isoparaffin (ISOPAR E), C10-13 alkane (ACUTEMP LP1820), 011-13 isoparaffin, C12-17 alkane (IRIS), C13-15 alkane (GEMSEAL 25), C15-19 alkane (EMOGREEN L19, EMOGREEN L15, GEMSEAL 40), C14-22 alkane (LILAC), 018-21 alkane (EMOSMART V21, VEGELIGHT 1820), C13-16 isoparaffin, coconut alkanes (VEGELIGHT 14), hydrogenated polyisobutene (C16-20), and their mixture, eg. PERMETHYL 216C, PERMETHYL 222C, PERMETHYL 296C. In some exemplary embodiments, the fatty compound may include fatty alcohol chosen from undecanol, octyldodecanol, hexydecanol. In some embodiments, the at least one fatty compound is present in an amount of from about 20 to about 60 wt. % based on the total weight of the cosmetic composition. In some exemplary embodiments the at least one fatty compound is chosen from alkanes having from 10 through 30 carbons.

In some cases, the cosmetic composition can include at least one surfactant. In some embodiments, the at least one surfactant is selected from monoxyalkylenated or polyoxyalkylenated nonionic surfactants, monoglycerolated or polyglycerolated nonionic surfactants, alkylpolyglucosides, and combinations thereof. In one embodiment, the at least one surfactant is selected steareth-2, steareth-10, steareth-20, steareth-21, steareth-25, steareth-100, cetyl alcohol, ceteth-2, ceteth-20, stearyl alcohol, ceteareth-20, ceteareth-12, ceteareth-25, cetearyl alcohol, cetearyl glucoside, laureth-7, laureth-23, and combinations thereof. In an exemplary embodiment, a cosmetic composition may include the at least one surfactant in an amount from about 0.5 to about 10 wt. % based on the total weight of the cosmetic composition.

In some cases, the cosmetic composition can include at least one oxidizing agent stabilizer. In accordance with the various embodiments, the at least one oxidizing agent stabilizer is selected from sodium phytate, sodium stannate, tetrasodium pyrophosphate, trisodium ethylenediamine disuccinate, ethylenediaminetetraacetic acid, tetrasodium etidronate, tetrasodium glutamate diacetate, pentasodium ethylenediamine tetramethylene phosphonate, sodium hexametaphosphate and combinations thereof. In some embodiments, the at least one oxidizing agent stabilizer is present from about 0.01 to about 1 wt. % based on the total weight of the cosmetic composition.

In some cases, the cosmetic composition includes a polymer. Non-limiting examples of polymers include fatty acid amides, cellulose-based thickeners, carboxylic acid polymers, cross-linked polyacrylate polymers, polyacrylamide polymers, polysaccharides, gums, and combination thereof.

Additionally, or alternatively, the cosmetic composition includes a preservative including sodium salicylate.

In some cases, the cosmetic composition includes a vitamin or vitamin derivative selected from ascorbic acid, tocopherol and combinations thereof.

In some embodiments, the cosmetic composition includes a solvent including water.

In accordance with the various embodiments, a cosmetic composition is in a form selected from a suspension, cream, serum, essence, gel, toner, stick, spray, ointment, paste, foam, mousse, shaving cream, wipe, strip, film-forming product, facial masks and skin masks.

The methods of the disclosure are useful for desquamation of the skin; and are useful for improving the pore size appearance, reducing casual sebum, improving radiance of the skin, improving the evenness of skin tone, and improving the overall appearance of the skin.

Another aspect of the instant disclosure can include a cosmetic composition comprising:
a. At least one oxidizing agent;
b. at least one fatty compound present from about at least 20 wt. %, the said fatty compound having a molecular weight of less than about 400 g/mol;
c. optionally at least one surfactant; and
d. optionally at least one oxidizing agent stabilizer; and
wherein the cosmetic composition has a pH lower than 7; and
wherein the composition can accelerate skin rejuvenation, reduce skin pore size appearance and casual sebum, improve skin tone evenness and radiance, and improve overall skin appearance; and
wherein all percentages by weight are based on the total weight of the cosmetic composition.

Another aspect of the instant disclosure can include a cosmetic composition comprising:
a. from about 1 to about 4 wt. % of an oxidizing agent comprising hydrogen peroxide;
b. from about 20 to about 60 wt % of at least one fatty compound selected from C15-19 Alkane, C13-16 Isoparaffin, Isohexadecane and combinations thereof; the said fatty compound having a molecular weight of less than 400 g/mol;
c. from about 0.5 to about 8% of at least one surfactant; and
d. from about 0.1 to about 1 wt. % of at least one oxidizing agent stabilizer;
wherein the cosmetic composition has a pH<7;
wherein the composition can accelerate skin rejuvenation, reduce skin pore size appearance and casual sebum, improve skin tone evenness and radiance, and improve overall skin appearance; and wherein all percentages by weight are based on the total weight of the cosmetic composition.

In another exemplary embodiment, a method for exfoliating the skin includes applying to the skin a composition comprising:
a. an oxidizing agent;
b. at least one fatty compound present from about at least 20 wt. %, the said fatty compound having a molecular weight of less than 400 g/mol;
and wherein the composition can accelerate skin rejuvenation, reduce skin pore size appearance and casual sebum, improve skin tone evenness and radiance, and improve overall skin appearance.

In another exemplary embodiment, a method for exfoliating skin includes applying to the skin a composition comprising:
a. an oxidizing agent;
b. at least one fatty compound present from about at least 20 wt. %, the said fatty compound having a molecular weight of less than 400 g/mol;
c. at least one surfactant; and
d. at least one oxidizing agent stabilizer; and

wherein the cosmetic composition has a pH lower than 7; and

wherein the composition can accelerate skin rejuvenation, reduce skin pore size appearance and casual sebum, improve skin tone evenness and radiance, and improve overall skin appearance; and wherein all percentages by weight are based on the total weight of the cosmetic composition.

More specifically, the methods of the instant disclosure include applying a cosmetic composition to the skin and allowing it to remain on the skin for a period of time. The composition may be allowed to remain on the skin for a period of time. The period of time may be, for example, of at least 1 min, then the skin is rinsed off. In some various embodiments, the methods describe herein provide exfoliation of the skin, improve the radiance of skin; improves the evenness of skin tone; improves the pore size appearance of the skin; reduces casual sebum level; and/or improves the overall appearance of skin.

Other features and advantages of the present invention will be apparent from the following more detailed description, by way of example, the principles of the invention.

This disclosure describes exemplary embodiments in accordance with the general inventive concepts and is not intended to limit the scope of the invention in any way. Indeed, the invention as described in the specification is broader than and unlimited by the exemplary embodiments set forth herein, and the terms used herein have their full ordinary meaning.

### BRIEF DESCRIPTION OF THE DRAWINGS

Implementations of the present technology will now be described, by way of example only, with reference to the attached figures, wherein:

FIG. 1 graphically shows the shifts of the melting transition temperatures measured by Differential Scanning calorimetry (DSC) associated with treatment-induced disordering of intercellular lipids in stratum corneum;

FIG. 2 graphically shows and compares the lipid disorganization results;

FIG. 3 graphically shows the improvement of the facial pore size appearance, skin radiance and tone evenness, and overall skin appearance, and forehead casual sebum.

It should be understood that the various aspects are not limited to the arrangements and instrumentality shown in the Figures.

### DETAILED DESCRIPTION OF THE DISCLOSURE

Cosmetic compositions according to an aspect of the disclosure typically include:
a. At least one oxidizing agent;
b. at least one fatty compound present from about at least 20 wt. %, the said fatty compound having a molecular weight of less than about 400 g/mol; and
wherein the composition can accelerate skin rejuvenation, reduce skin pore size appearance and casual sebum, improve skin tone evenness and radiance, and improve overall skin appearance; and
wherein all percentages by weight are based on the total weight of the cosmetic composition.

The term “rejuvenation”, “exfoliation” or “desquamation” used herein are used interchangeably and refers to the skin renewal process through the specification of the application. Skin rejuvenation is often referred as skin's natural renewal process: new keratinocyte cells are continuously synthesized at the basal layer of skin, moving upwards to skin surface and eventually shed unnoticeably. The natural skin rejuvenation process typically takes around a month and slows down as skin aging.

### Oxidizing Agent

The oxidizing agent in the cosmetic composition, according to the disclosure in some embodiments, is discovered to be compatible with cosmetic applications. In some embodiments, the composition may comprise one or more of hydrogen peroxide, urea peroxide, carbamide peroxide, and PVP hydrogen peroxide (a complex of polyvinylpyrrolidone and hydrogen peroxide), and combination thereof.

In accordance with the various embodiments, amount of oxidizing agent present in the composition can range from about 1% to about 4%, from about 1 to about 3.8%, from about 1.0 to about 3.6%, from about 1.0 to about 3.5%, from 1.2 to about 3.5%, from 1.2 to about 3.2% or any suitable combination, sub-combination, range, or sub-range thereof by weight, based on the weight of the composition. One of ordinary skill in the art, however, will appreciate that other ranges are within the scope of the invention. In some embodiments, the oxidizing agent is hydrogen peroxide.

Thus, at least one oxidizing agent may be present, by weight, based on the total weight of the composition, each present from about 1.0, 1.2, 1.3, 1.4, 1.5, 1.6, 1.7, 1.8, 1.9, 2.0, 2.1, 2.2, 2.3, to about 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0 wt. %, based on the total weight of the cosmetic composition.

### Fatty Compound

In accordance with the disclosure, one or more fatty compound or oil having a molecular weight of less than about 400 g/mol is present in the composition.

In accordance with some of the various embodiments, the at least fatty compound is selected from low molecular linear or branched alkane (C6-21 alkane): isohexadecane, C8-9 isoparaffin (ISOPAR E), C10-13 alkane (ACUTEMP LP1820), C11-13 isoparaffin, C12-17 alkane (IRIS), C13-15 alkane (GEMSEAL 25), C15-19 alkane (EMOGREEN L19, EMOGREEN L15, GEMSEAL 40), C14-22 alkane (LILAC), C18-21 alkane (EMOSMART V21, VEGELIGHT 1820), C13-16 isoparaffin, coconut alkanes (VEGELIGHT 14), hydrogenated polyisobutene (C16-20), and their mixture, eg. PERMETHYL 216C, PERMETHYL 222C, PERMETHYL 296C. In some exemplary embodiments, the fatty compound may include fatty alcohol chosen from undecanol, octyldodecanol, hexydecanol. In some embodiments, the at least one fatty compound is present in an amount of from about 20 to about 60 wt. % based on the total weight of the cosmetic composition. In some exemplary embodiments the at least one fatty compound is chosen from alkanes having from 10 through 30 carbons.

In some embodiments, the at least one fatty compound is an organic compound that is insoluble in water at ordinary ambient temperature (25° C.) and at atmospheric pressure (760 mmHg). In some embodiments, the at least one fatty substance has a water solubility of less than 5%. In some embodiments, the at least one fatty compound has a water solubility of less than 1%. In some embodiments, the at least one fatty substance has a water solubility of less than 0.1%. Although these fatty compounds are given as an example, it will be appreciated that other compounds compatible with cosmetic applications known in the art may be used.

In accordance with the various embodiments, amounts of the fatty compounds present in the composition can range from about 20% to about 60%, or from about 25 to about 60%, from about 30 to about 59%, from about 35 to about 58%, from about 40 to about 57%, from about 42 to about 56%, from about 30 to about 55%, from about 30 to about 60% or any suitable combination, sub-combination, range, or sub-range thereof by weight, based on the weight of the composition. In some embodiments according to the disclosure, the composition includes at least about 20% of the fatty compound. And, in some embodiments according to the disclosure, the composition includes at least about 50% of the fatty compound. Thus, in some embodiments, the fatty compound is present in an amount that. One of ordinary skill in the art, however, will appreciate that other ranges are within the scope of the invention. In some embodiments, two or more fatty compounds are present.

Thus, any one of or a combination of fatty compounds may be present, by weight, based on the total weight of the composition, each one or the combination present from about 20, 21, 22, 23, 24, 25, 26, 27, 28, 29, 30, 31, 32, 33, 34, 35, 36, 37, 38, 39, 40 to about 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or about 80 wt. %, based on the total weight of the cosmetic composition.

### Surfactant

The cosmetic compositions of the instant disclosure may optionally include at least one surfactant. The amount of the at least one surfactant in the cosmetic compositions, if present, is typically from about 0.5% to about 10% by weight relative to the weight of the composition.

The at least one surfactant may be selected from nonionic surfactants and anionic surfactants. In some embodiments, the composition includes one, two, three or more surfactants. In some exemplary embodiments, the surfactant or surfactants are nonionic. In some embodiments, the at least one surfactant includes monoxyalkylenated or polyoxyalkylenated nonionic surfactants, monoglycerolated or polyglycerolated nonionic surfactants, alkylpolyglucoside nonionic surfactants, or combinations thereof. In some embodiments, the composition includes at least one nonionic surfactant. In some embodiments, the composition includes two or more surfactants, and in some such embodiments, at least one surfactant has a low HLB (about 5) and at least one surfactant has a high HLB (about 15).

Exemplary anionic surfactants include, but are not limited to, the salts (in particular alkali metal salts, for example, sodium salts, amine salts such as aminoalcohol salts or alkaline-earth metal salts such as magnesium salts) of the following compounds: alkyl sulfates, alkyl ether sulfates, alkylamido ether sulfates, alkylaryl polyether sulfates, monoglyceride sulfates; alkylsulfonates, alkylamidesulfonates, alkylarylsulfonates, α-olefin sulfonates, paraffin sulfonates; alkyl phosphates, alkyl ether phosphates; alkylsulfosuccinates, alkyl ether sulfosuccinates, alkylamidesulfosuccinates; alkylsulfosuccinamates; alkylsulfoacetates; acylsarcosinates; acylisethionates and N-acyltaurates; salts of fatty acids such as oleic acid, ricinoleic acid, palmitic acid or stearic acid, coconut oil acid or hydrogenated coconut oil acid; alkyl-D-galactoside uronic acid salts; acyllactylates; salts of polyoxyalkylenated alkyl ether carboxylic acids, of polyoxyalkylenated alkylaryl ether carboxylic acids or of polyoxyalkylenated alkylamido ether carboxylic acids, in particular those having from 2 to 50 ethylene oxide groups; and combinations thereof.

Exemplary nonionic surfactants include, but are not limited to, monooxyalkylenated or polyoxyalkylenated nonionic surfactants, monoglycerolated or polyglycerolated nonionic surfactants, or alkylpolyglucosides. The oxyalkylene units may be oxyethylene or oxypropylene units, or a combination thereof. In some embodiments, the oxyalkylene units are oxyethylene units. Exemplary oxyalkylenated nonionic surfactants include, but are not limited to: oxyalkylenated (C8-C24) alkylphenols, saturated or unsaturated, linear or branched, oxyalkylenated C8-C30 alcohols, saturated or unsaturated, linear or branched, oxyalkylenated C8-C30 amides, esters of saturated or unsaturated, linear or branched, C8-C30 acids and of polyethylene glycols, polyoxyethylenated esters of saturated or unsaturated, linear or branched, C8-C30 acids and of sorbitol, saturated or unsaturated, oxyethylenated plant oils, condensates of ethylene oxide and/or of propylene oxide, inter alia, alone or as a mixture.

Exemplary alkylpolyglucosides include, but are not limited to, decyl glucoside, caprylyl/capryl glucoside, laurylglucoside, coco-glucoside, cetostearyl glucoside possibly mixed with cetostearyl alcohol, arachidyl glucoside, cocoylethylglucoside, and a mixture thereof.

In some embodiments, the at least one surfactant present in the composition is a nonionic surfactant. In some exemplary embodiments, one or more surfactants in the composition are selected from oxyalkylenated (C8-C24) alkylphenols, saturated or unsaturated, linear or branched, oxyalkylenated C8-C30 alcohols, saturated or unsaturated, linear or branched, oxyalkylenated C8-C30 amides, esters of saturated or unsaturated, linear or branched, C8-C30 acids and of polyethylene glycols, polyoxyethylenated esters of saturated or unsaturated, linear or branched, C8-C30 acids and of sorbitol, saturated or unsaturated, oxyethylenated plant oils, condensates of ethylene oxide and/or propylene oxide and combinations thereof. In some exemplary embodiments a composition according to the disclosure includes two or more of nonionic surfactants selected from oxyethylenated C8-C30 alcohols, polyoxyethylenated linear or branched, saturated or unsaturated C8-C30 acid esters, and polyoxyethylenated sorbitol esters.

In some embodiments, the composition includes at least one surfactant selected from steareth 2, steareth-10, steareth-20, steareth-21, steareth-25, steareth-100, cetyl alcohol, ceteth-2, ceteth-20, stearyl alcohol, ceteareth-20, ceteareth-12, ceteareth-25, cetearyl alcohol, cetearyl glucoside, laureth-7, laureth-23, and combinations thereof.

In some embodiments, the composition includes two or more surfactants, and in some such embodiments, at least one surfactant has a low HLB (about 5), and in the range from about 1-8, more preferably 3≤HLB≤8, and at least one surfactant has a high HLB (about 15) and in the range from about 8-25, more preferably 10≤HLB≤16. In accordance with such embodiments, at least one surfactant has an HLB in the range from about 1 to about 8, for example from about 1, 2, 3, 4, 5, 6, 7, or 8, and at least one surfactant has an HLB in the range from about 8 to about 25, for example, from about 8, 9, 10, 11, 12, 13, 14, 15, 16, 17, 18, 19, 20, 21, 22, 23, 24 or 25. In some exemplary embodiments, a composition includes one or more of CETETH-2 (HLB˜5) and STEARETH-2 (HLB˜5), and one or more of DECYL GLUCOSIDE (HLB˜13-15) and STEARETH-20 (HLB˜15).

In some embodiments, the at least one surfactant, if present in the composition, is in an amount ranging from about 0.5% to about 10% by weight relative to the weight of the composition. In some embodiments, the at least one surfactant, if present, in the composition in an amount ranging from about 0.8% to about 8% by weight, including increments and ranges therein and there between, based upon the total weight of the composition.

In some embodiments, one or more surfactants, alone or in combination, can be present in the composition according to the disclosure, and in some embodiments, each surfactant may be present from about 0.5% to about 10% by weight, from about 1.0% to about 8.0% by weight, from about 1.5% to about 5.0%, from about 0.5 to about 4.5%, and from about 0.5 to about 4.0%, or any suitable combination, sub-combination, range, or sub-range thereof by weight, based on the weight of the composition.

Thus, one or a combination of surfactants may be present, by weight, based on the total weight of the composition, each one or the combination present from about 0.5, 0.6, 0.7, 0.8, 0.9, 1.0, 2, 3, 4, 5, 6, 7, 8, 9, or about 10 wt. %, based on the total weight of the cosmetic composition.

### Oxidizing Agent Stabilizer

In some embodiments, at least one or more oxidizing agent stabilizer may optionally be present in the composition according to the disclosure from about 0.01% to about 1% by weight, from about 0.02% to about 0.9% by weight, from about 0.02% to about 0.85%, from about 0.02% to about 0.8%, and from about 0.025 to about 0.75%, or any suitable combination, sub-combination, range, or sub-range thereof by weight, based on the weight of the composition. In some exemplary embodiments, oxidizing agent stabilizer are selected from sodium phytate, sodium stannate, tetrasodium pyrophosphate, trisodium ethylenediamine disuccinate, ethylenediaminetetraacetic acid, tetrasodium etidronate, tetrasodium glutamate diacetate, pentasodium ethylenediamine tetramethylene phosphonate, sodium hexametaphosphate and combinations thereof.

Thus, one or a combination of oxidizing agents may be present, by weight, based on the total weight, each one or the combination present from about 0.01, 0.02, 0.03, 0.04, 0.05, 0.06, 0.07, 0.08, 0.09 to about 0.09, 0.1, 0.2, 0.3, 0.4, 0.5, 0.6, 0.7, 0.8, 0.9, or about 1.0 wt. %, based on the total weight of the cosmetic composition.

### Polymer

In some embodiments, one or more other components, such as polymers can be present in the composition according to the disclosure from about 0.05% to about 5% by weight, from about 0.05% to about 3% by weight, from about 0.1 to about 3%, from about 0.25 to about 1%, and from about 0.25 to about 0.5%, or any suitable combination, sub-combination, range, or sub-range thereof by weight, based on the weight of the composition. In some embodiments, polymers are selected from fatty acid amides, cellulose-based thickeners, carboxylic acid polymers, cross-linked polyacrylate polymers, polyacrylamide polymers, polysaccharides, gums, and a mixture thereof. In some exemplary embodiments, polymers are selected from cetyl hydroxyethylcellulose, sclerotium gum at 1% or more by weight, and combinations of these.

Thus, one or a combination of polymers may be present, by weight, based on the total weight of the composition, each one or the combination present from about 0.05, 0.06, 0.07, 0.08, 0.09, 0.1, 0.2, 0.3, 0.4, 0.5, 0.6, 0.7, 0.8, 0.9, 1.0, 2, 3, 4, 5 weight percent, including increments and ranges therein and there between.

### Solvent

In accordance with the disclosure, one or more solvent is present in the composition. The solvent present in the cosmetic composition, according to the disclosure, includes, but is not limited to, water, alcohol, propylene glycol, or combinations thereof. Although these solvents are given as an example, it will be appreciated that other solvents compatible with cosmetic applications known in the art may be used.

In accordance with the various embodiments, solvent is present in a given composition in an amount of from about 1% to about 70%, or any suitable combination, sub-combination, range, or sub-range thereof by weight, based on the weight of the composition. One of ordinary skill in the art, however, will appreciate that other ranges are within the scope of the invention

Thus, one or a combination of solvents may be present, by weight, based on the total weight of the composition, each one or the combination present from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70 weight percent, including increments and ranges therein and there between.

Water

The compositions comprise from about 0 to about 70% by weight of water, with respect to the total weight of the composition. In some embodiments, the amount of water in the composition can range from about 1 to about 50%, or any suitable combination, sub-combination, range, or sub-range thereof by weight, based on the weight of the composition.

The pH of the composition is not limited but is generally below 7, and in some embodiments is one of between 2 and 7, and between 2 and 6.5, and between 2.5 and 6.5, and 7. The pH can be adjusted to the desired value by addition of a base (organic or inorganic) to the composition, for example ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid, phosphoric acid.

Thus, water may be present by weight, based on the total weight of the composition, from about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, to about 70 weight percent, including increments and ranges therein and there between.

### Optional Components

In some embodiments, there may be one or more actives present in the cosmetic composition, according to the disclosure, the additive selected from, for example, humectants, such as acetamide MEA, glycols, such as glycerin and propylene glycol; alcohol; anti-microbial components, salicylic acid, alpha acid; antioxidants, including, but not limited to, phenolic compounds, such as chalcones, flavones, flavanones, flavanols, flavonols, dihydroflavonols, isoflavonoids, neoflavonoids, catechins, anthocyanidins, tannins, lignans, aurones, stilbenoids, curcuminoids, alkylphenols, betacyanins, capsacinoids, hydroxybenzoketones, methoxyphenols, naphthoquinones, and phenolic terpenes, resveratrol, curcumin, pinoresinol, ferulic acid, hydroxytyrosol, cinnamic acid, caffeic acid, p-coumaric acid, baicalin (Scutellaria Baicalensis root extract), pine bark extract (Pinus Pinaster bark/bud extract), ellagic acid; and vitamins and vitamin derivatives, such as tocopherol and ascorbic acid; and combinations thereof.

In some embodiments, there may be one or more other components present in the cosmetic composition, according to the disclosure, the other components selected from, fillers such as clays, talc, organic thickeners with for instance, anionic, cationic, nonionic, and amphoteric polymeric associative thickeners and combinations thereof; penetrants; sequestrants; fragrances; dispersants; film-forming agents; ceramides; opacifiers and combinations thereof. Although the aforementioned optional components are given as an example, it will be appreciated that other optional components compatible with cosmetic applications known in the art may be used.

In accordance with the various embodiments, the amount of actives and other components present in the composition can range from about 0 to about 50%, from about 0.5 to about 30%, from about 1.5 to about 20%, and from about 5 to about 15%, or any suitable combination, sub-combination, range, or sub-range thereof by weight, based on the weight of the composition.

In some embodiments, one or more actives, alone or in combination, can be present in the composition according to the disclosure from about 0.05 to about 50% by weight, from about 0.05% to about 2.5% by weight, from about 0.1 to about 2%, from about 0.25 to about 1.5%, and from about 0.5 to about 1.25%, or any suitable combination, sub-combination, range, or sub-range thereof by weight, based on the weight of the composition.

In some embodiments, one or more other components, such as preservatives, vitamins, preservatives, and the like, alone or in combination, can be present in the composition according to the disclosure from about 0.05 to about 50% by weight, from about 0.05% to about 25% by weight, from about 0.1 to about 10%, from about 0.25% to about 5%, and from about 0.5 to about 3.5%, or any suitable combination, sub-combination, range, or sub-range thereof by weight, based on the weight of the composition. In some exemplary embodiments, preservatives may include sodium salicylate, and vitamins may include ascorbic acid, tocopherol and combinations of these.

Thus, one or a combination of optional components may be present, by weight, based on the total weight of the composition, each one or the combination present from about 0.05, 0.06, 0.07, 0.08, 0.09, 0.1, 0.2, 0.3, 0.4, 0.5, 0.6, 0.7, 0.8, 0.9, 1.0, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 to about 50 weight percent, including increments and ranges therein and there between.

Methods

The methods of the instant disclosure include a method for exfoliating skin, comprising applying to the skin a composition comprising:
a. an oxidizing agent;
b. at least one fatty compound present from about at least 20 wt. %, the said fatty compound having a molecular weight of less than 400 g/mol;

and
wherein the composition can accelerate skin rejuvenation, reduce skin pore size appearance and casual sebum, improve skin tone evenness and radiance, and improve overall skin appearance; and
wherein all percentages by weight are based on the total weight of the cosmetic composition.

More specifically, the methods of the instant disclosure include applying a cosmetic composition to the skin and allowing it to remain on the skin for a length of time. The composition may be allowed to remain on the skin for a period of time. The period of time may be, for example, of at least 1 minutes, then the skin is rinsed off.

The length of time that the cosmetic composition is allowed to remain on the skin can vary. Nonetheless, as noted above, these compositions are usually allowed to remain on the skin for at least 1 minutes, and may be allowed to remain on the skin for longer periods of time, for example they may be allowed to remain on the skin for up to 15 minutes without rinsing or washing. The cosmetic composition is then rinse or wash from the skin.

In some instances, the cosmetic composition can be applied to the skin and allowed to remain on the skin for about 1 minute to about 15 minutes, about 1 minute to about 8 minutes, from about 1 minute to about 7 minutes, from about 1 minute to about 6 minutes, from about 1 minutes to about 5 minutes, from about 1 minutes to about 4 minutes, about 1 minute to about 3 minutes or from about 1 minutes to about 2 minutes before rinsing the composition from the skin.

The methods for treating skin are particularly useful for treating the skin of the face and neck, in particular, in human individuals. The methods provide for exfoliation of the skin, which can result in the improvement of skin surface appearance, such as skin radiance, brightness, tone evenness, hyperpigmentation, pore appearance, fine lines/wrinkles, etc.

The method improves the radiance of skin; improves the evenness of skin tone; reduces the pore size appearance of the skin; reduces the casual sebum level of the skin; and improves the overall appearance of skin.

Implementation of the present disclosure is provided by way of the following examples. The examples serve to illustrate the technology without being limiting in nature.

### EXAMPLES

Implementation of the present disclosure is provided by way of the following examples. The following examples serve to elucidate aspects of the technology without being limiting in nature.

### Example 1

### Inventive Compositions

TABLE 1
Inventive Compositions

| | | Inv. Ex. 1 | Inv. Ex. 2 | Inv. Ex. 3 | Inv. Ex. 4 | Inv. Ex. 5 |
|---|---|---|---|---|---|---|
| a | HYDROGEN PEROXIDE | 2.0 | 2.0 | 1.0 | 2.0 | 2.0 |
| b | C15-19 ALKANE (low MW) | 50.0 | 25.0 | 50.0 | 50.0 | 50.0 |
| | C13-16 ISOPARAFFIN (Low MW) | 0 | 25.0 | 0 | 0 | 0 |
| c | STEARETH-2 | 3.5 | 3.5 | 3.5 | 3.5 | 3.5 |
| c | STEARETH-20 | 1.5 | 1.5 | 1.5 | 1.5 | 1.5 |
| d | SODIUM PHYTATE | 0 | 0 | 0 | 0.1 | 0 |
| d | TETRASODIUM GLUTAMATE | 0 | 0 | 0 | 0 | 0.1 |
| | DIACETATE | | | | | |
| d | TETRASODIUM PYROPHOSPHATE | 0.2 | 0.2 | 0.2 | 0.0 | 0.0 |
| d | TRISODIUM ETHYLENEDIAMINE | 0.1 | 0.1 | 0.1 | 0 | 0 |
| | DISUCCINATE | | | | | |
| | BUFFERING AGENT | 0.2 | 0.2 | 0.2 | 0.2 | 0.2 |
| | POLYMER | 1.0 | 1.0 | 1.0 | 1.0 | 1.0 |
| | pH ADJUSTER | 0.1 | 0.2 | 0.1 | <0.1 | 0.1 |
| | PRESERVATIVE | <0.1 | <0.1 | <0.1 | <0.1 | <0.1 |
| | VITAMIN | 0.2 | 0.2 | 0.2 | 0.2 | 0.2 |
| | WATER | Q.S. | Q.S. | Q.S. | Q.S. | Q.S. |

TABLE 2
Comparative Examples

| | | Comparative | Comparative |
|---|---|---|---|
| Claims | INCI Names | Ex. 1 | Ex. 2 |
| a | HYDROGEN PEROXIDE | 2.0 | 2.0 |
| b | C15-19 ALKANE | 0 | 0 |
| | (low MW) | | |
| | C13-16 ISOPARAFFIN | 0 | 0 |
| | (Low MW) | | |
| | ISOPROPYL MYRISTATE | 0 | 1.0 |
| | MINERAL OIL | 50.0 | 0 |
| | (high MW) | | |
| c | STEARETH-2 | 3.5 | 0 |
| c | STEARETH-20 | 1.5 | 0 |
| d | TETRASODIUM | 0.2 | 0.2 |
| | PYROPHOSPHATE | | |
| d | TRISODIUM | 0 | 0.1 |
| | ETHYLENEDIAMINE | | |
| | DISUCCINATE | | |
| d | PENTASODIUM | 0.1 | 0 |
| | ETHYLENEDIAMINE | | |
| | TETRAMETHYLENE | | |
| | PHOSPHONATE | | |
| | BUFFERING AGENT | 0.2 | 0 |
| | POLYMER | 1.0 | 1.5 |
| | pH ADJUSTER | <0.1 | <0.1 |
| | PRESERVATIVE | 0.05 | 0.05 |
| | VITAMIN | 0.2 | 0 |
| | WATER | Q.S. | Q.S. |

### Example 2

### Exfoliation in Vitro and in Vivo Performance

Various compositions were tested and compared to determine which compositions produced the greatest degree of lipid disorganization, which is a common method for estimating the desquamation efficacy. All DSC experiments were performed on isolated human stratum corneum (SC). The SC was prepared using a standard protocol. Human abdominal skin remaining from plastic surgery was obtained in frozen form. Subcutaneous fat was removed and the dermis was separated from epidermis by plunging the skin into hot water (56° C.) for 1-2 min. The remaining viable epidermis was removed by tryptic digestion at 37° C. overnight. The SC was dried and stored at room temperature and ambient humidity. Samples of stratum corneum were equilibrated at 75% relative humidity (RH), at room temperature (RT) at least overnight before testing. The following five protocols were tested:
1. Positive Control: A solution of 70% glycolic acid (GA) was tested as a positive control.
2. Comparative Ex. 1 (containing 2% H2O2 and high molecular weight oil=Mineral Oil) was tested.
3. Inventive Ex. 1 (containing 2% H2O2 and low molecular weight oil=C15-19 ALKANE) was tested.
4. Comparative Ex. 2 (containing 2% H2O2 in gel and no oil) was tested.
5. Inventive Ex. 2 (containing 2% H2O2 and a mixture of low molecular weight oil=C15-19 ALKANE+C13-16 ISOPARAFFIN) was tested.
6. Inventive Ex. 3 (containing 1% H2O2 and a low molecular weight oil=C15-19 ALKANE) was tested.

0.1 gram of each of a test composition was applied to a sample of stratum corneum. The test composition was allowed to remain on the stratum corneum for 15 minutes at 75% RH, at RT, after which the surface of the stratum corneum was wiped clean, rinsed with deionized water and tab dry with tissue paper.

The intercellular lipids in stratum corneum are organized in highly ordered multilayers where lipid aliphatic chains are predominantly arranged in a crystalline structure. This tight packing of lipid molecules ensures the barrier function of the stratum corneum. Any treatment that reduces lipid ordering will potentially impair the barrier function, thus promoting the penetration of topically applied substances through the SC. The degree of lipid disorganization following treatment can be conveniently monitored by the shifts of the melting transition temperatures measured by Differential Scanning Calorimetry (DSC). The thermogramme of the untreated stratum corneum is characterized by 4 phase transitions:

T1—around 40° C. corresponding to the change of lipid chain packing from orthorhombic to hexagonal;

T2—around 72° C. corresponding in the melting of alkyl chains;

T3—around 85° C. corresponding to the disturbance of the lamellar arrangement of lipids and loss of ordering at the level of polar heads (this peak is also often attributed to comeocyte-bound lipids);

T4—above 110° C. (irreversible) corresponding to the denaturation of proteins¹.

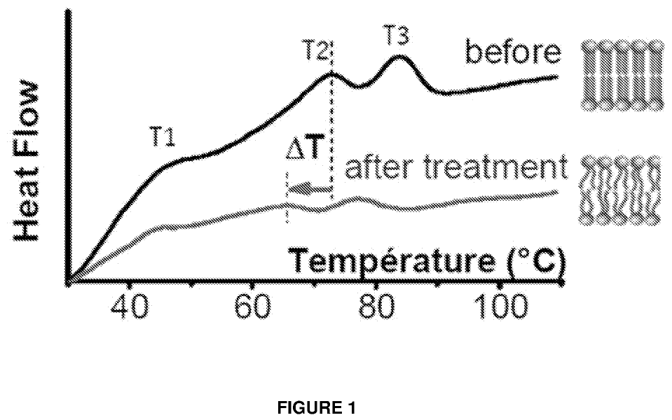

The endothermic peaks corresponding to transitions T1, T2 and T3 for a typical SC sample are shown in FIG. 1.

Shifts in the lipid phase transition temperatures of T2 and T3 following treatment are interpreted as evidence of product penetration into the SC, interaction with intercellular lipids and disordering (fluidizing) effect on their crystal organization. Lipid disordering on the molecular level is translated into a deterioration of the barrier properties of the SC at the tissue level. A decrease of >=2° C. is considered significant. In addition, if the shift of melting temperature is associated with a drop of transition enthalpy (the area under the peak), this can be interpreted as a partial loss (extraction) of the lipids during treatment. The measurements were performed using DSC Q200 (TA Instruments) using Tzero aluminium hermetic pans and heating from 20° to 120° C. at a rate of 5° C./min. An empty pan was used as a reference.

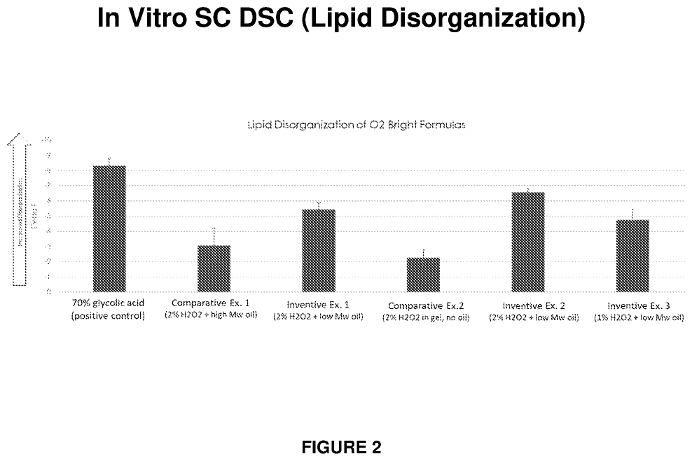

The maxima of the endothermic peaks corresponding to lipid phase transitions are determined using dedicated software (TA Universal Analysis). The peak position and the transition enthalpy are determined with respect to a manually defined linear baseline. Specific enthalpy is calculated using the hydrated (75% RH) SC mass before treatment. The shifts of the melting peak positions (ΔTm(T2) and ΔTm(T3)) are calculated with respect to reference thermogrammes obtained with untreated or vehicle-treated samples. For simplicity, an effective shift is calculated as a linear combination of the measured shifts for the T2 and T3 transitions for each treatment: ΔTeffective=0.7·ΔTm(T2)+0.3·ΔTm(T3). Higher weight is given to the T2 transition as it is our primary criterion of lipid perturbation with well-established interpretation; at the same time, the T3 contribution is also included. The results are reported in the table below (Table 3) and graphically presented in FIG. 2.

TABLE 3

| | | Compositions | ΔT | Std |
|---|---|---|---|---|
| 1 | Positive | 70% GA | −8.32 | 0.52 |
| | Control | | | |
| 2 | Comparative | 2% H2O2 and | −3.07 | 1.13 |
| | Example 1 | Mineral Oil | | |
| 3 | Inventive | 2% H2O2 and | −5.43 | 0.48 |
| | Example 1 | C15-19 ALKANE | | |
| 4 | Comparative | 2% H2O2 in | −2.27 | 0.5 |
| | Example 2 | gel and no oil | | |
| 5 | Inventive | 2% H2O2 and | −6.58 | 0.20 |
| | Example 2 | a mixture of C15-19 | | |
| | | ALKANE + C13-16 | | |
| | | ISOPARAFFIN | | |
| 6 | Inventive | 1% H2O2 and | −4.75 | 0.70 |
| | Example 3 | C15-19 ALKANE | | |

The positive control used was a formula containing 70% of glycolic acid which is known to highly disrupt the lipid organization of the Stratum Corneum. Several formulas were tested and compared. Treatment with composition containing only 2% H2O2 had basically no influence on exfoliation. A significant level of lipid disruption was only observed with Examples containing 1% or 2% H2O2 and a low molecular weight oils (i.e., C15-C19 branched alkane (Mw˜250 g/mol). That was the case for Inventive Example 1, Inventive Example 2 and Inventive Example 3. See FIG. 2.

It was observed that Comparative Ex. 1 containing 2% H2O2 but contained high molecular weight oil (i.e. mineral oil (Mw˜450 g/mol)) didn't show the same lipid disorganization and allowed to conclude that the molecular weight of the oils was an essential factor on the exfoliation of the skin.

Comparative Ex. 2 containing 2% H2O2 without any oil demonstrated no additional lipid disorganization.

A clinical study on volar forearm minizones was performed to evaluate the efficacy of Inventive Ex. 1 (i.e., 2% H2O2 in 50% C15-C19 branched alkane (Mw˜250 g/mol)) in comparison to 3.8% lactic acid peel serum and untreated control on skin exfoliation. Thirty-one female subjects aged 24 to 64 years completed the study. The volar forearm minizones were treated with Dansyl Chloride (DC) patches for 24 hours. After the patches were removed, the presence of DC fluorescence on each minizone was graded using a Wood's Lamp Light. The Inventive Ex. 1 exhibited similar skin exfoliation rates as the 3.8% lactic acid peel serum and showed significant acceleration of exfoliation (p<0.05) compared to the untreated control at D21 (Table 4).

The results are shown in Table 4

TABLE 4
Dansyl Chloride Total Fluorescence Scores

| | Day 3 | Day 5 | Day 7 | Day 14 | Day 21 |
|---|---|---|---|---|---|
| | Inventive Composition | | | | |
| Mean | 13.73 | 21.40 | 28.87 | 49.09 | 59.37 |
| Std. Dev. | 0.76 | 1.51 | 2.36 | 5.68 | 7.62 |
| P-value vs Untreated | 0.12 | 0.08 | 0.09 | 0.06 | 0.03* |
| P-value vs 3.8% Lactic Add | 0.08 | 0.12 | 0.24 | 0.28 | 0.07 |
| | 3.8% Lactic Acid | | | | |
| Mean | 13.92 | 21.72 | 29.21 | 49.73 | 60.97 |
| Std. Dev. | 0.75 | 1.52 | 2.33 | 5.2 | 6.69 |
| A. P-value vs Untreated | 0.69 | 0.76 | 0.48 | 0.32 | 0.67 |
| | Untreated | | | | |
| Mean | 13.90 | 21.78 | 29.42 | 50.35 | 61.31 |
| Std. Dev. | 0.76 | 1.47 | 2.22 | 4.68 | 5.76 |

The results above allowed to conclude surprisingly that the low molecular weight of the oil associated with the H2O2 had an impact on the increase of the lipid disorganization and had an impact on the exfoliation of the skin.

### Example 3

### In Vivo Performance on Pore Size Appearance, Skin Tone Evenness, Skin Radiance, Overall Skin Appearance, and Casual Sebum

A clinical study on thirty volunteers with Fitzpatrick Type I-V and mild to moderate skin blemishes was performed in order to characterize the improvement of the pore size appearance, casual sebum, skin radiance and tone evenness, and overall skin appearance.

The inventive composition (2% H2O2+Low Mw oil fla) was tested by applying it on a full face twice per week, leaving it on for 5 min on the skin, and then rinsing it off, over the course of 56 days. A modified Griffith scale with 0-9 scores was used for clinical grading of facial pore size appearance, skin tone evenness, skin radiance, overall skin appearance. A Sebumeter® SM 815 (Courage & Khazaka) was used to measure casual sebum level on the forehead.

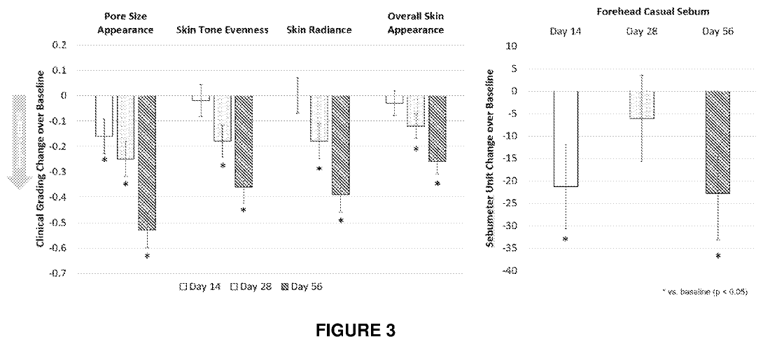

Compared to baseline, the inventive composition treatment showed statistically significant reduction (p<0.05) in pore size appearance with clinical grading changes of 0.16, 0.25, and 0.53 at Days 14, 28, and 56, respectively. The inventive composition treatment showed statistically significant improvement (p<0.05) in skin tone evenness, skin radiance, and overall skin appearance with clinical grading changes of 0.18 and 0.36, 0.18 and 0.39, and 0.12 and 0.26, respectively, at Days 28 and 56. The inventive composition treatment also showed statistically significant reduction (p<0.05) in forehead casual sebum with changes of 21.36 and 22.83 sebumeter units at Days 14 and 56, respectively. (FIG. 3)

The above results demonstrated the efficacy of the inventive compositions on the facial pore size appearance and forehead casual sebum reduction, as well as facial skin tone evenness, skin radiance, and overall skin appearance improvement with repeated application over time. These benefits potentially resulted from the skin exfoliation property of the inventive composition.

The term “INCI” is an abbreviation of International Nomenclature of Cosmetic Ingredients, which is a system of names provided by the International Nomenclature Committee of the Personal Care Products Council to describe personal care ingredients.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

All components and elements positively set forth in this disclosure can be negatively excluded from the claims. In other words, the cosmetic compositions of the instant disclosure can be free or essentially free of all components and elements positively recited throughout the instant disclosure.

Some of the various categories of components identified may overlap. In such cases where overlap may exist and the composition includes both components (or the composition includes more than two components that overlap), an overlapping compound does not represent more than one component. For example, a fatty acid may be characterized as both a nonionic surfactant and a fatty compound. If a particular composition includes both a nonionic surfactant and a fatty compound, a single fatty acid will serve as only the nonionic surfactant or as only the fatty compound (the single fatty acid does not serve as both the nonionic surfactant and the fatty compound).

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The expression “one or more” means “at least one” and thus includes individual components as well as mixtures/combinations.

Other than in the operating examples, or where otherwise indicated, all numbers expressing quantities of ingredients and/or reaction conditions may be modified in all instances by the term “about,” meaning within +/−5% of the indicated number.

The term “treat” (and its grammatical variations) as used herein refers to the application of the compositions of the present disclosure onto the surface of keratinous substrates such as skin, in particular, the skin of the head, face, and neck.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. All of the components set forth herein may be optionally included or excluded from the compositions/method/kits. When excluded, the compositions/methods/kits may be free or essentially free of the component. For example, a particular composition may be free or essentially free of alkoxylated compounds, for example, ethoxylated thickeners and/or ethoxylated surfactants. Likewise, a particular composition may be free or essentially free of sulfates, such as sulfate surfactants.

What is claimed:

1. A cosmetic composition comprising:
a. At least one oxidizing agent;
b. at least one fatty compound present from about at least 20 wt. %, the said fatty compound having a molecular weight of less than about 400 g/mol; and
wherein the composition can accelerate skin rejuvenation, reduce skin pore size appearance and casual sebum, improve skin tone evenness and radiance, and improve overall skin appearance; and
wherein all percentages by weight are based on the total weight of the cosmetic composition.

2. The composition of claim 1 without the presence of an activator for the at least one oxidizing agent.

3. The composition of claim 1, wherein the composition, when applied at least once per week to keratinous tissue accelerate skin rejuvenation.

4. The composition of claim 1, wherein the at least one oxidizing agent is present in an amount of from about 1 to about 4 wt. % based on the total weight of the cosmetic composition.

5. The composition of claim 1, wherein the at least one oxidizing agent is chosen from hydrogen peroxide, urea peroxide, carbamide peroxide, metal peroxide, and PVP hydrogen peroxide (a complex of polyvinylpyrrolidone and hydrogen peroxide), metal peroxide and combinations thereof.

6. The composition of claim 1, wherein the at least one fatty compound is selected from low molecular linear or branched alkane; isohexadecane; C8-9 isoparaffin; C10-13 alkane; C11-13 isoparaffin; C12-17 alkane; C13-15 alkane; C15-19 alkane; C14-22 alkane; C18-21 alkane; C13-16 isoparaffin, coconut alkanes; hydrogenated polyisobutene (C16-20), undecanol, octyldodecanol, hexydecanol and combinations thereof.

7. The composition of claim 1, wherein the at least one fatty compound is present in an amount of from about 20 to about 60 wt. % based on the total weight of the cosmetic composition.

8. The composition of claim 1, further comprising at least one surfactant.

9. The composition of claim 8, wherein the at least one surfactant is selected from monoxyalkylenated or polyoxyalkylenated nonionic surfactants, monoglycerolated or polyglycerolated nonionic surfactants, alkylpolyglucosides, and combinations thereof.

10. The composition of claim 9, wherein the at least one surfactant is selected steareth 2, steareth-10, steareth-20, steareth-21, steareth-25, steareth-100, cetyl alcohol, ceteth-2, ceteth-20, stearyl alcohol, ceteareth-20, ceteareth-12, ceteareth-25, cetearyl alcohol, cetearyl glucoside, laureth-7, laureth-23, and combinations thereof.

11. The composition of claim 1, further comprising at least one oxidizing agent stabilizer.

12. The composition of claim 1, wherein the at least one oxidizing agent stabilizer is selected from sodium phytate, sodium stannate, tetrasodium pyrophosphate, trisodium ethylenediamine disuccinate, ethylenediaminetetraacetic acid, tetrasodium etidronate, tetrasodium glutamate diacetate, pentasodium ethylenediamine tetramethylene phosphonate, sodium hexametaphosphate and combinations thereof.

13. The composition of claim 1 further comprising a polymer selected from fatty acid amides, cellulose-based thickeners, carboxylic acid polymers, cross-linked polyacrylate polymers, polyacrylamide polymers, polysaccharides, gums, and combinations thereof.

14. The composition of claim 1 further comprises a preservative including sodium salicylate.

15. The composition of claim 1 further comprises a solvent including water.

16. A method for improving the appearance of the skin comprising applying a composition of claim 1 to the skin.

17. A method for exfoliating skin, comprising applying to the skin a composition comprising:
a. an oxidizing agent;
b. at least one fatty compound present from about at least 20 wt. %, the said fatty compound having a molecular weight of less than 400 g/mol; and
wherein the composition can accelerate skin rejuvenation, reduce skin pore size appearance and casual sebum, improve skin tone evenness and radiance, and improve overall skin appearance; and
wherein all percentage by weight are based on the total weight of the cosmetic composition.

18. The method of claim 17, wherein the method:
improves the pore size appearance of the skin;
reduces the casual sebum level on skin surface;
improves the evenness of skin tone;
improves the radiance of skin;
improves the overall appearance of skin.

19. The method of claim 18, wherein the method provides exfoliation of the skin.
